(19) United States
(12) Patent Application Publication
(10) Pub. No.: US 2026/0051384 A1
(43) Pub. Date: Feb. 19, 2026

(54) METHODS AND SYSTEMS FOR DETERMINING FLOW MODELS AND INFUSION RATES FOR APHERESIS SYSTEMS

(71) Applicant: Terumo BCT, Inc., Lakewood, CO (US)

(72) Inventors: Cindie MCLEAN, Lakewood, CO (US); Craig Ruff, Boulder, CO (US); Jerry Bill, Lakewood, CO (US); John Lindner, Lakewood, CO (US); Scott Butzke, Littleton, CO (US)

(21) Appl. No.: 18/808,172

(22) Filed: Aug. 19, 2024

(51) Int. Cl. G16H 20/17; A61M 1/02

(52) U.S. Cl. CPC G16H 20/17; A61M 1/0259

(57) ABSTRACT

A method for determining a flow model for fluid within an apheresis machine includes determining a starting flow model for an apheresis procedure, determining details of a secondary device, and generating an updated flow model based on the starting flow model and the details of the secondary device. The secondary device is configured to connect to the apheresis machine for the apheresis procedure.

### FIELD

The present disclosure relates to systems and methods for determining fluid flow models for apheresis systems. The present disclosure also relates to systems and methods for determining an infusion rate of an anticoagulant solution during an apheresis procedure.

### BACKGROUND

This section provides background information related to the present disclosure which is not necessarily prior art.

Therapeutic apheresis systems are designed to collect cells from a source or donor and to treat a patient. A single machine may be used for both treatment and collection. During collection, whole blood may be collected, followed by a centrifugal process that separates blood components from the whole blood based on the density of the blood component. During treatment, a patient may be hooked up to the therapeutic apheresis system to receive one or more blood components. At least initial fluid flow models and infusion rates may be determined prior to initiating an apheresis treatment.

### BRIEF SUMMARY

This section provides a general summary of the disclosure and is not a comprehensive disclosure of its full scope or all of its features.

At least one example embodiment is a method for determining a flow model for fluid within an apheresis machine. The method may include determining a starting flow model for an apheresis procedure, determining details of a secondary device, and generating an updated flow model based on the starting flow model and the details of the secondary device. The secondary device may be configured to connect to the apheresis machine for the apheresis procedure.

In at least one example embodiment, the secondary device may be an external plasma treatment device.

In at least one example embodiment, determining the starting flow model may include determining a fluid volume through each fluid line of a plurality of fluid lines of the apheresis machine, determining a fluid volume in each fluid chamber of a plurality of fluid chambers of the apheresis machine, and determining a plurality of fluids utilized during the apheresis procedure.

In at least one example embodiment, determining details of the secondary device may include receiving a volume of the secondary device and determining an unknown fluid component within the secondary device when the secondary device is connected to the apheresis machine.

Also described herein is a method for performing an apheresis procedure using a flow model. The method may include connecting a patient to an apheresis machine for the apheresis procedure, the apheresis machine including a secondary device, generating a flow model for use during the apheresis procedure, and initiating the apheresis procedure. The flow model may incorporate the apheresis machine and the secondary device.

In at least one example embodiment, the secondary device may be an external plasma treatment device.

In at least one example embodiment, generating the flow model may include determining a starting flow model for the apheresis procedure, determining details of the secondary device, and generating an updated flow model based on the starting flow model and the details of the secondary device. In at least one example embodiment, determining the starting flow model may include determining a fluid volume through each fluid line of a plurality of fluid lines of the apheresis machine, determining a fluid volume in each fluid chamber of a plurality of fluid chambers of the apheresis machine, and determining a plurality of fluids utilized during the apheresis procedure. In at least one example embodiment, determining details of the secondary device may include receiving a volume of the secondary device and determining an unknown fluid component within the secondary device when the secondary device is connected to the apheresis machine.

In at least one example embodiment, the method may further include tracking an amount of heparin throughout the apheresis machine and the secondary device during the apheresis procedure.

In at least one example embodiment, the method may further include outputting a status of the apheresis procedure during operation of the apheresis machine.

Also described herein is a method for adjusting a ratio of inlet patient blood to anticoagulant (AC) during an apheresis procedure. The method may include determining a citrate molarity for a first interval of a plurality of intervals, during a current interval after the first interval, determining a citrate molarity for a previous interval, determining if the citrate molarity for the previous interval is greater than a constant multiplied by the citrate molarity of the first interval and increasing the ratio of inlet patient blood to AC in response to the citrate molarity for the previous interval being greater than the constant multiplied by the citrate molarity of the first interval.

In at least one example embodiment, the method may further include maintaining the ratio of inlet patient blood to AC in response to the citrate molarity for the previous interval being less than or equal to the constant multiplied by the citrate molarity of the first interval.

In at least one example embodiment, the constant may be determined based on a time between intervals of the plurality of intervals.

In at least one example embodiment, the citrate molarity for the previous interval is determined by dividing a sum of a number of moles of citrate in the inlet patient blood and a number of moles of citrate of the AC by a volume of an apheresis system divided by a change in time from a previous interval. In at least one example embodiment, the number of moles of citrate in the inlet patient blood may be determined by multiplying a change in volume of the inlet patient blood from a previous interval by a plasma fraction and by a citrate molarity of the patient. In at least one example embodiment, the number of moles of citrate of the AC may be determined by multiplying a change in volume of the AC from a previous interval by a citrate molarity of the AC. In at least one example embodiment, the apheresis system may include fluid components of an apheresis machine and blood components of the patient.

In at least one example embodiment, the AC may be ACD-A that includes citrate ions with a presumed half-life of 80 minutes.

In at least one example embodiment, the ratio of inlet patient blood to AC may be set by an operator prior to initiating the apheresis procedure.

### BRIEF DESCRIPTION OF THE SEVERAL VIEWS OF THE DRAWINGS

The drawings described herein are for illustrative purposes only of selected embodiments and not all possible implementations and are not intended to limit the scope of the present disclosure.

FIG. 1 is a front view of an apheresis machine according to an example embodiment.

FIG. 2 is a block diagram of a system of the apheresis machine of FIG. 1 suitable for implementing the methods described herein according to an example embodiment.

FIG. 3 is a front view of fluid lines and components of the apheresis machine of FIG. 1 according to an example embodiment.

FIG. 4 is a flow chart of a method for determining a flow model according to an example embodiment.

FIG. 5 is a flow chart of the step 402 of determining a starting flow model of FIG. 4 according to an example embodiment.

FIG. 6 is a flow chart of the step 404 of determining details of a secondary device of FIG. 4 according to an example embodiment.

FIG. 7 is a flow chart of a method of performing an apheresis procedure according to an example embodiment.

FIG. 8 is a method of adjusting a ratio of inlet patient blood to anticoagulant during an apheresis procedure according to an example embodiment.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (I) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

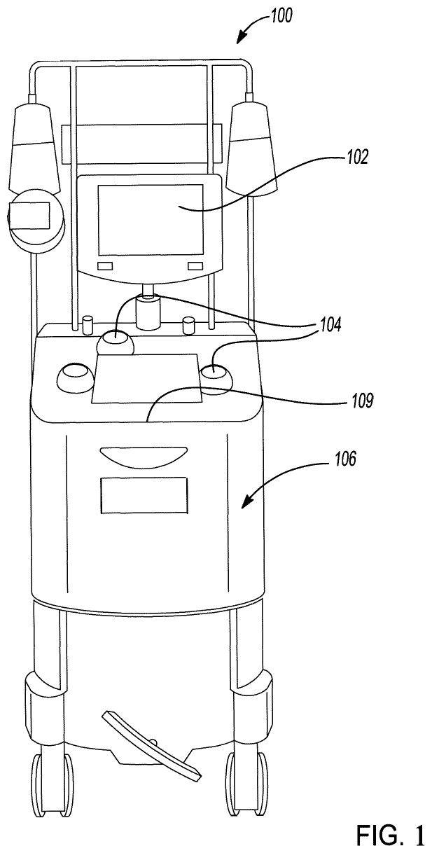

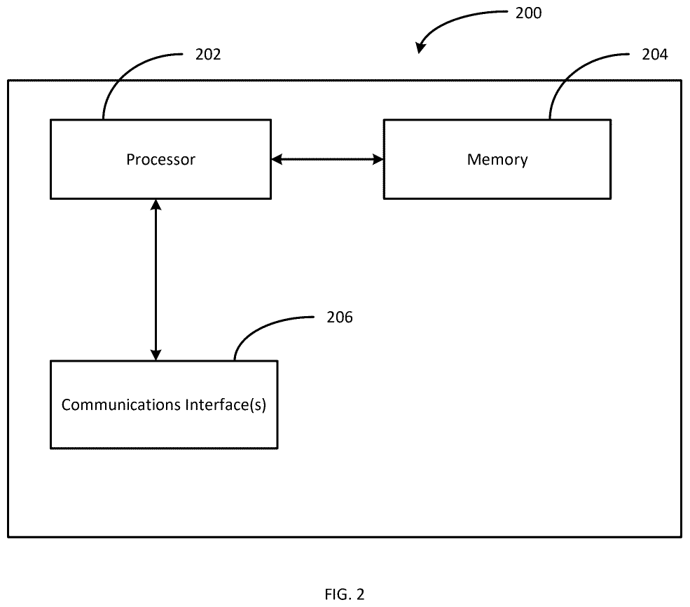

FIG. 1 show an example embodiment of an apheresis machine 100. The apheresis machine 100 may be utilized in apheresis procedures and may have components configured to connect to a patient or user to complete an apheresis procedure. The components of the apheresis machine 100 are described in further detail below with respect to FIG. 2.

The apheresis machine 100 may additionally include a user interface 102. The user interface 102 may be configured to display information about the patient or user as well as information about the apheresis procedure being completed by the apheresis machine 100. The apheresis machine 100 may additionally include one or more pumps 104 that may be managed by software of the apheresis machine 100. Further details of the one or more pumps 104 are described below.

FIG. 2 is a block diagram of a system 200 that may be included in the apheresis machine 100. The system 200 may be a computer or other architecture capable of performing the methods and functionality described herein. The system 200 may include at least one processor 202. The at least one processor 202 may be a central processing unit (CPU) or other suitable processor(s). The system 200 may further include a memory 204 such as a random access memory (RAM), read only memory (ROM), or another suitable memory.

The system 200 also may include one or more input/output devices 206. The one or more input/output devices 206 may include a user input device, such as a keyboard, a keypad, a mouse, and the like, a user output device, such as a display, a speaker, and the like, an input port, an output port, a receiver, a transmitter, one or more storage devices, such as a tape drive, a floppy drive, a hard disk drive, a compact disk drive, and the like, as well as various combinations thereof. The methods and processes described herein will be described with respect to the system 200.

### Flow Model

A flow model is used to determine flows and volumes for an apheresis procedure. There may be a plurality of flow models for a particular apheresis machine and/or procedure. Traditional apheresis flow models do not account for third party connections to the apheresis machine that may alter flows and/or volumes during an apheresis procedure. Embodiments described herein incorporate third party data and equipment to enable a more accurate flow model to be generated for a particular apheresis machine and/or procedure.

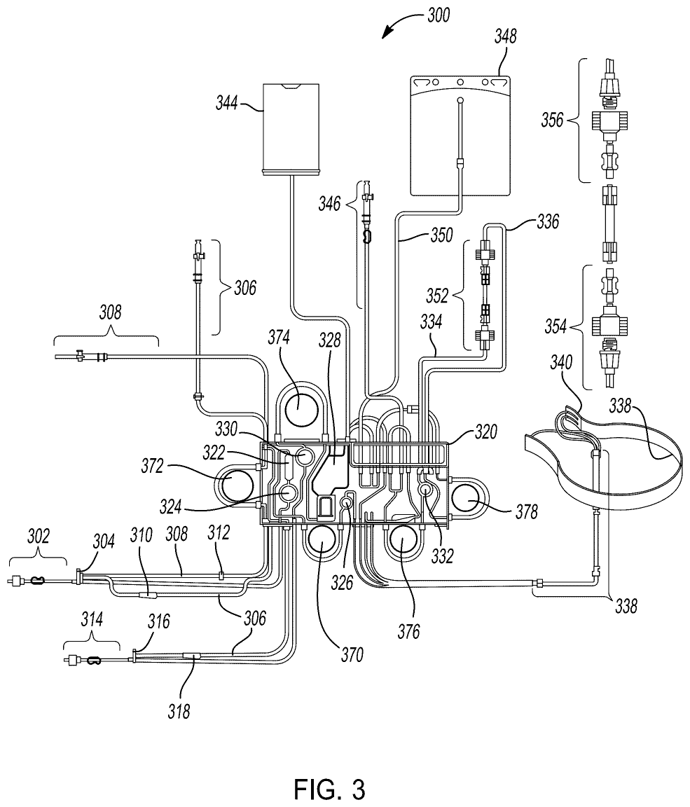

FIG. 3 is a diagram of fluid components 300 of the apheresis machine 100. The fluid components 300 may include the internal components of the apheresis machine 100 described above. The fluid components 300 may also be or may include disposable portions of the apheresis machine 100. Disposable portions of the apheresis machine 100 may be elements that may be removed and disposed of via proper channels and may be replaced with new components. Any portion of the fluid components 300 that houses fluid may be a disposable. Thus, these portions may be discarded and replaced for any new patient or donor. The disposables may be primed and prepared for use each time they are replaced for each new patient and/or donor. The fluid components 300 may be analyzed and volumes and/or flows through the fluid components 300 may be incorporated into a fluid model for an apheresis procedure. As described herein, the fluid components 300 may interact with the one or more pumps 104. The one or more pumps 104 are schematically shown in FIG. 3 within a loop of the particular fluid line configured to interact with the particular pump.

In at least one example embodiment, the fluid components 300 may include an inlet line 302 that may be coupled to an inlet line manifold 304. The inlet line 302 may be coupled to a patient or donor or source during an apheresis procedure. The inlet line manifold 304 may be coupled to each of the inlet line 302, a saline line 306, and an anticoagulant (“AC”) line 308. The saline line 306 may include a first saline line clamp 310 and the AC line 308 may include an AC check valve 312, each of which are configured to control fluid flow through the saline line 306 and the AC line 308, respectively. The inlet line 302 may be configured to interact with an inlet pump 370. The inlet pump 370 may be configured to restrict and/or permit fluid flow through a portion of the inlet line 302 that is interacting with the inlet pump 370. The AC line 308 may be configured to interact with an AC pump 372. The AC pump 372 may be configured to restrict and/or permit fluid flow through a portion of the AC line 308 that is interacting with the AC pump 372.

The fluid components 300 may further include a return line 314. The return line 314 may be configured to return fluid back to the patient or donor after the fluid has made its way through the various fluid components of the apheresis machine 100. Similar to the inlet line 302, the return line 314 may include a return line manifold 316. The return line manifold 314 may be coupled to both the return line 314 and the saline line 306. The saline line 306 may additionally include a second saline line clamp 318 which may be configured to control fluid flow through the saline line 306. The return line line 314 may be configured to interact with a return pump 374. The return pump 374 may be configured to restrict and/or permit fluid flow through a portion of the return line 314 that is interacting with the return pump 374.

Each of the inlet line 302, the return line 314, the saline line 306, and the AC line 308 may be configured to intersect with a cassette 320. The cassette 320 may include in inlet line trap 322, an inlet pressure sensor diaphragm 324, a centrifuge pressure sensor diaphragm 326, a reservoir 328, a return line pressure sensor diaphragm 330, and a plasma pressure sensor diaphragm 332. Each of the inlet pressure sensor diaphragm 324, the centrifuge pressure sensor diaphragm 326, the return line pressure sensor diaphragm 330, and the plasma pressure sensor diaphragm 332 may be configured to sense a pressure within each of the respective fluid lines or elements of the apheresis machine 100. For example, the inlet pressure sensor diaphragm 324 may be configured to indicate a pressure within the inlet line 302, the centrifuge pressure sensor diaphragm 326 may be configured to indicate a pressure within a centrifuge of the apheresis machine 100, the return line pressure sensor diaphragm 330 may be configured to indicate a pressure within the return line 314, and the plasma pressure sensor diaphragm 332 may be configured to indicate a pressure within at least one of a plasma inlet line 334 or a treated plasma line 336. In at least one example embodiment, the plasma inlet line 334 may be configured to interact with a plasma pump 376. The plasma pump 376 may be configured to restrict and/or permit fluid flow through a portion of the plasma inlet line 334 that is interacting with the plasma pump 376.

One or more of the inlet line 302, the return line 314, the saline line 306, the AC line 308, the plasma inlet line 334, or the treated plasma line 336 may be configured to couple between the cassette 320 and a centrifuge via a centrifuge loop 338. The centrifuge loop may interact with a channel 340 and a connector 342 of the centrifuge.

The fluid components 300 may further include a vent bag 344 that may be configured to interact with the cassette 320. The vent bag 344 may provide fluid into the fluid components in certain apheresis procedures.

The fluid components 300 may also include a plasma device fluid line 346. The plasma device fluid line 346 may be used to prime a secondary plasma device (“SPD”) with a fluid other than saline in at least one example embodiment. In at least one example embodiment, the SPD may be an external plasma treatment device. The terms secondary plasma device, SPD, and external plasma treatment device may be used interchangeably herein. In at least one example embodiment, the plasma device fluid line 346 may be configured to interact with an SPD pump 378. The SPD pump 378 may be configured to move fluid through the SPD during an apheresis procedure.

The fluid components 300 may further include a waste bag 348 and a waste bag line 350. The waste bag 348 may be connected to the cassette 320 via the waste line 350. The waste bag 348 may be configured to collect waste produced and/or collected within the fluid components during an apheresis procedure.

The fluid components 300 may additionally include one or more connections configured to couple elements of the fluid components 300 to external elements. In particular, the fluid components 300 may include plasma device connections 352, plasma inlet connections 354, and treated plasma connections 356. each of the plasma device connections 352, the plasma inlet connections 354, and the treated plasma connections 356 may be luer connectors in at least one example embodiments. Alternatively, or additionally, the connections may be another fluid tight connection.

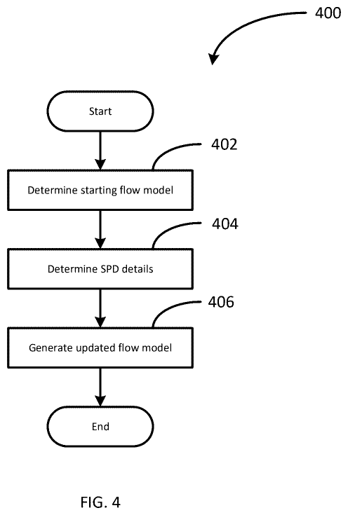

FIG. 4 is a flow chart of a method 400 of determining a flow model. The method 400 is described below with reference to the fluid components 300 of FIG. 3 and the system 300 of FIG. 2. However, example embodiments are not limited herein.

The method 400 begins with the processor 202 determining a starting flow model. In at least one example embodiment, there may be a default flow model that is selected until a user input is received via the one or more input/output devices 206 which defines the particular apheresis procedure being initiated. The default flow model is not used for an apheresis procedure and is only a placeholder until a particular apheresis procedure is defined and initiated. Once a user input is received, a particular flow model may be initiated by the processor 202. In at least one example embodiment, the particular flow model may correspond to a starting state of the fluid components 300 of the apheresis machine 100. Both the default flow model and the particular flow model may only include components native to the apheresis machine 100. Thus, any components of a third party that may be hooked up to the apheresis machine 100 are excluded from the flow model. The flow model represents a current state of the fluid components of the apheresis machine 100 and may be used for control and safety systems of the apheresis machine 100. Thus, example embodiments herein describe improved flow models that incorporate third party information to provide a complete flow model for improved used in at least control and safety systems of the apheresis machine 100. Additional details of the starting flow model are described below with reference to FIG. 5.

In at least one example embodiment, the component from a third party that may be attached to the apheresis machine 100 may be an SPD. The SPD may be configured to treat plasma received from a patient during an apheresis procedure. In at least one example embodiment, the SPD may be configured to remove one or more targeted components. The SPD may include one or more columns, filters, and/or external devices that may be designed by a third party for a particular apheresis procedure. Details of the SPD are input by the operator into the flow model after the details are received from a manufacturer of the SPD.

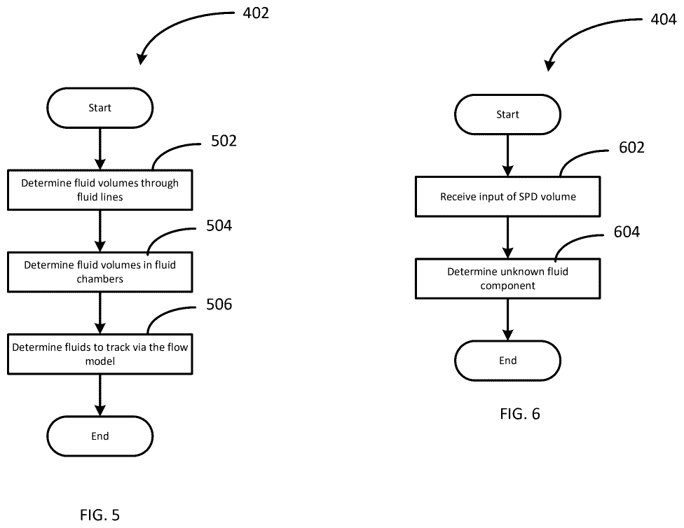

At step 404, the processor 202 determines details of the SPD. In at least one example embodiment, the details of the SPD may be received from an operator. The operator may use the one or more input/output devices 206 to provide the details of the SPD or the details may be communicated to the apheresis machine 100 in a different method such as wireless communication, for example. Further details of the details of the SPD provided by an operator are discussed below with reference to FIG. 6.

At step 406, the processor 202 generates an updated flow model by incorporating the details of the SPD into the starting flow model. The updated flow model may incorporate the details of the SPD such that component fractions are correctly modeled to ensure patient safety requirements are met for an apheresis procedure. Incorporation of details of the SPD also ensures that an accurate procedure status display may be output to the operator during an apheresis procedure. In particular, the SPD adds a volume to the flow path. Thus, all calculations of the flow model need to incorporate the volume of the SPD for accuracy. Ensuring an accurate volume of all of the fluid components 300 including the disposables and the SPD ensures that the flow model is properly modeled to ensure accuracy and safety. Thus, the updated flow model may correspond to an updated state of a system including the apheresis machine 100 and the SPD.

FIG. 5 is a flow chart of determining the starting flow model of the step 402 of FIG. 4. At step 502, a total volume through the fluid lines of the apheresis machine 100 is determined and incorporated into the flow model by the processor 202. The starting flow model may include a total accumulated volume through each line of the fluid components 300. Thus, the flow model may include a total accumulated volume through each of the inlet line 302, the saline line 306, the AC line 308, the return line 314, the plasma inlet line 334, the treated plasma line 336, the plasma device fluid line 346, and the waste bag line 250. In at least one example embodiment, the total accumulated volume through each of the lines described above includes a volume that flows through the lines as they interact with one or more pumps of the apheresis machine 100. For example, the inlet line 302 may interact with the inlet pump 370 and the flow model may incorporate the total accumulated volume within the inlet pump from the inlet line 302. Similarly, the return line 314 may interact with the return pump 374 both during priming of the apheresis machine 100 and during operation of the apheresis machine 100, the AC line 308 may interact with the AC pump 372, the plasma inlet line 334 may interact with the plasma pump 376, and one or more of the treated plasma line 336, or the plasma device fluid line 346 may interact with the SPD pump 378.

At step 504, the processor 202 determines fluid volumes within fluid chambers of the apheresis machine 100. In particular, a volume within the channel 340 and the reservoir 328 is determined and incorporated into the starting flow model by the processor 202.

At step 506, the processor 202 determines the various fluids that are to be tracked with the flow model during an apheresis procedure. In particular, the flow model may track each fluid that is utilized during an apheresis procedure. The various fluids that may flow through the apheresis machine 100 may include AC such as anticoagulant citrate dextrose solution, solution A (“ACD-A”) or ACD-A/heparin, heparin, untreated plasma, red blood cells (“RBC”), saline, and treated plasma. In at least one example embodiment, a volume of heparin may not be a significant volumetric component of the AC volume within the apheresis machine 100. Thus, the heparin content may be tracked in units per mL of the AC volume present in a particular chamber or fluid line of the apheresis machine 100. Modelling the heparin content in this manner allows the heparin concentration to change throughout an apheresis procedure.

FIG. 6 is a flow chart of the step 404 of determining details of the SPD. At step 602, the volume of the SPD may be input by an operator for a particular apheresis procedure. The volume may be input into the one or more input/output devices 206 by an operator in at least one example embodiment. The volume of the SPD may only be known to the operator as the SPD may be a secondary component that is attached to the apheresis machine 100. In at least one example embodiment, allowable flow rates through the SPD may also be input by the operator for the particular apheresis procedure. The allowable flow rates may be a flow rate or a range of flow rates that the SPD may be configured to accommodate during an apheresis procedure. In at least one example embodiment, the operator may additionally input a starting state of the SPD. For example, the SPD may be empty, primed with saline, or primed with another fluid. This information may be input by an operator for the particular apheresis procedure.

At step 604, an unknown fluid component is determined from the SPD. The unknown fluid component may be introduced to the apheresis machine from the SPD when the SPD is being primed by the apheresis machine 100 prior to initiating an apheresis procedure. The amount of fluid used to prime the SPD may be known and may be incorporated into the flow model by the processor 202. If the SPD is primed prior to connection to the apheresis machine 100, then the SPD is presumed to contain at least one of saline or another fluid. An operator may input an amount of saline used to prime the SPD or a standard amount is assumed and used as an input into the flow model. The amount of saline or another fluid may be related to the volume of the SPD that was input by the operator and a volume of the disposable elements of the fluid components 300. In particular, the amount of saline or another fluid is an amount that is sufficient to displace a volume of an unknown fluid content. In at least one example embodiment, an operator may input a larger volume than necessary as the amount of saline or other fluid within the SPD.

With the details of the SPD incorporated into the flow model, the flow model may provide a complete account of volumes and flow rates through all used components during an apheresis procedure. This may ensure that safety and control functions are accurate and provide a complete understanding of all components utilized during an apheresis procedure.

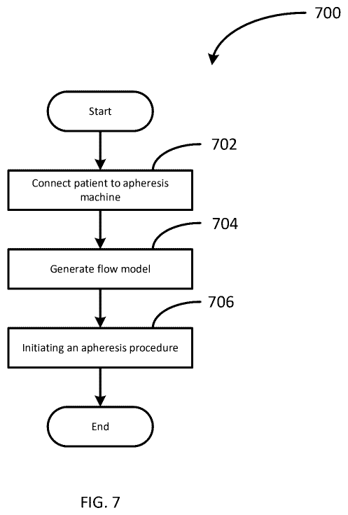

FIG. 7 is a method 700 of performing an apheresis procedure utilizing the flow model generated in FIGS. 4-6. At step 702, a patient may be connected to the apheresis machine. In at least one example embodiment, an operator may connect the patient to the apheresis machine by known methods in the art.

At step 704, a flow model is generated. The flow model may be generated as described above with reference to FIGS. 4-6.

At step 706, the processor 202 initiates an apheresis procedure using the apheresis machine 100. The flow model is used during an apheresis procedure to track volume and fractions of components within the apheresis machine. In at least one example embodiment, the flow model may be updated in regular intervals during an apheresis procedure. For example, the flow model may be updated every ten milliseconds. The interval may be greater or less than ten millisecond in example embodiments. In particular, the flow model have an initial starting state when the apheresis machine is empty. A first step in initiating the apheresis procedure may be to prime the disposable portions of the apheresis machine. Then, a patient and/or donor may be connected to the apheresis machine 100 for the apheresis procedure. During both the priming and the time during which the patient and/or donor is connected to the apheresis machine 100, the flow model may be updated. The flow model may be updated to incorporate additional information from the priming process and the process of connecting the patient and/or donor to the apheresis machine 100. As described above, the flow model may be updated every 10 ms in at least one example embodiment.

Use of the flow model provides various benefits such as preventing clotting of a patient's blood within the apheresis machine 100 at least based on use of the ACD-A anticoagulant. Further, patient safety may be maintained by preventing large fluid balance excursions of the patient's blood volume. The flow model may generate an output related to a status of the apheresis machine or the apheresis procedure such that an operator may be provided with useful information to monitor a patient. The outputs may be the state of the flow model after each 10 ms interval in at least one example embodiment. Providing an operator with this output may ameliorate side effects caused by citrate toxicity due to the use of the ACD-A anticoagulant. The flow model may also provide accurate end of procedure reporting. End of procedure reporting may include, without limitation, a volume of the patient's blood that was processed during the apheresis procedure, a volume of the patient's plasma that was treated, and any fluid balance changes or volumes of other fluids delivered to the patient during the apheresis procedure. In particular, the flow model may be used to track heparin that was administered during the apheresis procedure as a secondary anticoagulation factor to reduce risks of fluid balance increases.

### Infusion Rate

During apheresis procedures, an anticoagulant may be used to anticoagulate a patient's blood. The introduction of an anticoagulant may increase a patient's fluid volume because the apheresis machine, in conjunction with the patient's body, is a closed loop. However, an increase of the patient's fluid volume beyond a few percent is usually not desirable. In particular, longer running procedures, for example procedures that last about two to six hours may exacerbate an undesirable increase in a patient's fluid volume. Additionally, anticoagulant includes a citrate ion as an active component. When the anticoagulant is returned to a patient's body, the patient's body may be a source of citrate ion when additional blood is drawn into the disposable components of the fluid components 300. A patient's body may also metabolize or remove citrate ion over time as well as returning citrate ion to the disposable components. Accumulation of citrate ions within a patient's body may cause the patient to experience annoying, unpleasant or potentially life threatening side effects due to citrate ions binding to calcium ions causing citrate toxicity. To combat potential side effects from citrate ions, an operator of an apheresis machine may adjust either the infusion rate of the anticoagulant or a ratio of anticoagulant to the patient's blood. The example embodiments described herein describe an algorithm that is used to adjust an amount of ACD-A anticoagulant that is infused over time of an apheresis procedure.

The algorithm is used to track and update an estimate of a citrate molarity of a fluid volume during an apheresis procedure. In particular, the fluid volume consists of the fluid volume of the patient's body, an ACD-A container, and throughout the flow path of the apheresis machine. The flow path of the apheresis machine may include the fluid lines and fluid chambers of the fluid components 300 described above with respect to FIG. 3. The algorithm may model the citrate molarity of the patient's circulatory path, accounting for the removal of the citrate ion by the patient's body using a half-life decay rate. The algorithm may also use the patient's body as a secondary source of citrate being infused to the set via the blood being drawn by an inlet pump of the apheresis machine.

Before starting an apheresis procedure, an initial inlet blood to AC ratio is used to determine a citrate molarity that is presumed to be sufficient to maintain the apheresis machine's fluid paths in an anticoagulated state. The algorithm may then use a multiplier constant in conjunction with the initial citrate molarity value to determine a citrate molarity threshold when the inlet to AC ratio can be adjusted to lower the ACD-A being introduced from the ACD-A container.

The algorithm may be determined and/or executed with several underlying assumptions. First, the algorithm may assume that a standard ACD-A solution is the anticoagulant solution being used in the apheresis procedure. Next, the initial citrate molarity for effective anticoagulation is determined and/or set prior to beginning the apheresis procedure. In at least one example embodiment, an initial citrate molarity may not be sufficient for effective anticoagulation. If the initial citrate molarity is not sufficient for effective anticoagulation, an operator may adjust an infusion rate which may pause or end the algorithm.

Further, if a change is made to an inlet blood versus AC ratio during the apheresis procedure, the algorithm may be modified or a ramping feature controlled by the algorithm may be disabled. The ramping feature controlled by the algorithm may also be disabled if an upper limit of the inlet blood versus AC ratio is reached. Next, a half-life of 80 minutes for the AC is used for the algorithm. The half-life of 80 minutes is a half-life of the AC within a patient or donor body. This half-life may be used to account for patients who may have health conditions making their bodies less effective at citrate metabolism than the average person. Finally, the algorithm does not account for citrate ion exchange with a patient's interstitial fluid.

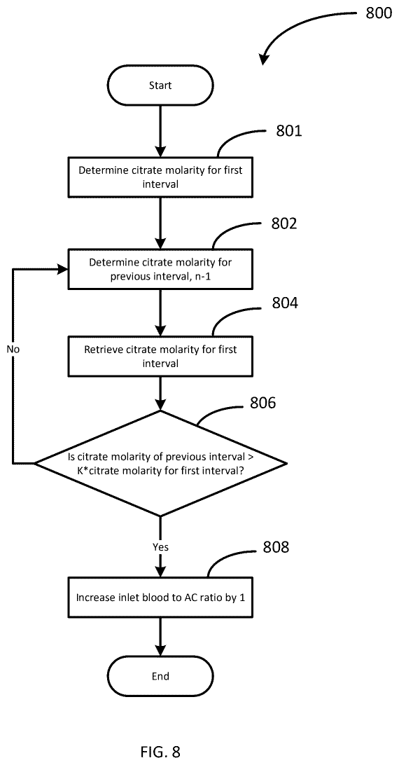

FIG. 8 is a flow chart of a method 800 of the algorithm used to track and update an estimate of a citrate molarity of a fluid volume during an apheresis procedure. An apheresis procedure may include a plurality of intervals. In at least one example embodiment, the algorithm may monitor and/or adjust the citrate molarity at each interval of the plurality of intervals. The algorithm may be executed in at least one example embodiment by the processor 202 of the apheresis machine.

At step 801, a citrate molarity of the first interval, n=1, is determined by the processor 202. The citrate molarity may be stored in the memory 204 in at least one example embodiment.

At step 802, a citrate molarity of a previous interval is determined by the processor 202. For example, if the current interval is n, then the previous interval is n−1. At interval n, the algorithm determines whether to adjust the citrate molarity by adjusting a ratio of inlet blood to AC. Further details of determining a citrate molarity are described below.

At step 804, a citrate molarity of the current interval, n, is determined by the processor 202. In at least one example embodiment, the step 802 and the step 804 may be interchangeable such that the citrate molarity of the current interval is determined prior to determining or retrieving the citrate molarity of the previous interval.

At conditional step 806, the processor 202 determines if the citrate molarity of the interval n−1 is greater than a constant multiplied by the citrate molarity of the first interval. If the citrate molarity of the interval n−1 is not greater than the constant multiplied by the citrate molarity of the first interval, then the method returns to step 802 for the next interval in the plurality of intervals. As shown in the method 800, the step 804 may be repeated for each interval. In at least one example embodiment, the citrate molarity of the first interval may be stored in the memory 204 and may be retrieved for each interval of the method 800 at step 804. For the first interval, there is not a previous interval to be retrieved. Thus, the citrate molarity of the previous interval does not exist and is not greater than a constant multiplied by the citrate molarity of the first interval. Therefore, at conditional step 806, the method 300 will follow the “no” path back to step 802 for a second interval.

If the citrate molarity of the interval n−1 is greater than the constant multiplied by the citrate molarity of the first interval, then at step 808 the processor increases the inlet blood to AC ratio by one.

In at least one example embodiment, an initial citrate molarity may be determined by first dividing an amount of citric acid by a molar mass of citric acid. Then dividing the result of the amount of citric acid divided by a molar mass of citric acid by a volume of the container housing the AC including the citric acid. For example, for a 750 mL bag of anticoagulant, such as a standard ACD-A solution, containing 21.9 g of citric acid, the citrate molarity of the anticoagulant may be

$${Citrate}_{ACDA} M = \frac{\frac{21.9 g}{192.124 g / mol}}{0.7 \; 50 L} = 0.1 \; 52 mol / L .$$

A patient's plasma volume may also need to be computed for execution of the algorithm. A patient's plasma volume may be computed from the values of a total blood volume and a extracellular fluid volume of the patient. In particular, plasma is generally about 3/14 of the total extracellular fluid volume of a patient.

Formulas used to compute a total blood volume and an extracellular fluid volume of a patient are generally known in the art. For example, Nadler's Formula may be used to determine a total blood volume given a known height and weight of the patient. Nadler's formula is well known in the art. However, it is known that one formula may not be optimal for both pediatric and non-pediatric patients. Thus, one formula may be used for non-pediatric patients and a second formula may be used for pediatric patients. For example, for patients below 25 kg in weight, the total blood volume of the patient must be manually entered rather than calculated by a formula. Further, one formula may be used for females and a separate formula may be used for males in at least one example embodiment. In at least one example embodiment, a common estimate for a total blood volume for a patient under 25 kg is 80 mL/kg.

An extracellular fluid volume for a patient may be estimated from a patient's weight in some embodiments. For example a volume of body fluid may be calculated by: V_{BodyFluid}(L)=0.6×weight (kg)×1 L/kg. Then a volume of extracellular fluid can be calculated by:

$${V}_{E C F} ( L ) = {V}_{B o d y F l u i d} ( L ) \times \frac{1}{3} ECF / BodyFluid .$$

There are other known methods of calculating an extracellular fluid volume known in the art.

From the total blood volume and the extracellular fluid volume, an effective plasma volume may be calculated by:

$${V}_{EffPlasma} ( L ) = max ( \frac{T B V ( mL ) \times plasmaFrac}{1000 mL / L} , {V}_{ECF} ( L ) \times \frac{3}{14} \frac{Plasma}{ECF} ) + {FluidBalance}_{c urrent} ( mL )$$

where TBV is the total blood volume, and plasmaFrac=1−HCT where HCT is a patient Hematocrit. The FluidBalance_{current} may be a delta volume of the patient during the apheresis procedure. For example, if 100 mL of fluid have been removed from the patient by the apheresis machine 100, then the FluidBalance_{current} would be −100 mL. The FluidBalance_{current} is the fluid balance at the time of the effective plasma volume calculation.

After the patient's plasma volume is determined, a citrate molarity of the patient can be determined at a point in time, tₙ₊₁, subsequent to a point in time, tₙ, where a state of the patient was known. First, a change in AC volume is determined by:

$${Δ}_{{V}_{A {C}_{n}}} ( L ) = \frac{{V}_{A {C}_{n + 1}} ( mL ) - {V}_{A {C}_{n}} ( mL )}{1000 mL / L} .$$

Then, a change in the patient's inlet blood volume is determined by:

$${Δ}_{{V}_{{PatientIn}_{n}}} ( L ) = \frac{{V}_{P a {tientIn}_{n + 1}} ( mL ) - {V}_{P a {tientIn}_{n}} ( mL )}{1000 mL / L} .$$

Then, an initial number of moles of citrate from AC is determined by: Citrate_{Infusion}_{ₙ} (mol)=Δ_{VAC}_{ₙ}(L)×Citrate_{ACDA}(M) and an initial number of modes of citrate from the patient is determined by: Citrate_{FromPatient}_{ₙ}(mol)=Δ_{V}_{_{PatientInn}} (L)×plasmaFrac×Citrate_{Patient}_{ₙ}(M) where plasmaFrac is a fraction of the patient's blood that is plasma. In at least one example embodiment, the fraction of the patient's blood that is plasma is simplified as determining the amount of the patient's blood that is not red blood cells. Thus, platelets, white blood cells, and other blood components are ignored. For example, a patient may have blood with 40% red blood cells and 60% plasma and would thus have a plasmaFrac of 0.6. Then the half-life is used to determine a number of modes remaining in the patient by:

$${Citrate}_{Remainin {g}_{n}} ( mol ) = {V}_{{EffPlasma}_{n}} ( L ) \times C i t r a t {e}_{{Patient}_{n}} ( M ) \times ( 1 - 0.5 \frac{{Δ}_{t} min}{{t}_{h a l f - l i f e} min} )$$

where Δt min=tₙ₊₁ min−tₙmin and t_{half-lif}emin=80 min. Finally, the citrate molarity of the patient at time tₙ₊₁ is determined by:

$${Citrate}_{{Patient}_{n + 1}} ( M ) = \frac{{Citrate}_{R e m a i n i n {g}_{n}} ( mol ) - {Citrate}_{{FromPatient}_{n}} ( mol ) + {Citrate}_{{Infusion}_{n}} ( mol )}{{V}_{{EffPlasma}_{n + 1}} ( L )}$$

In at least one example embodiment, a citrate molarity of the set is a normalized value determined by

$$Citr {ate}_{{Set}_{n}} ( M ) = \frac{{Citrate}_{{Infusion}_{n}} ( m o l ) + {Citrate}_{FromPatient} ( mol )}{{V}_{S e t} ( L ) / {Δ}_{t} min}$$

In at least one example embodiment, the algorithm is configured to control a ramping feature to adjust an amount of AC introduced during an apheresis procedure to adjust the inlet blood versus AC ratio. In particular, when the set citrate molarity calculated at an end of a previous interval is greater than a multiplier applied to the set citrate molarity of the first interval, the inlet blood versus AC ratio is increased by one: If (Citrate_{Set}_{_{n−1}}(M)(normalized)>K×Citrate_{Set}_{₁}(M) (normalized)), then Ratio (inlet blood:AC)=Ratio (inlet blood:AC)+1, where K is a constant multiplier. In at least one example embodiment, the value of K may be determined to cause the first ratio adjustment to occur at approximately t=20 min for a procedure performing in a steady state. For example, K=1.12 for a patient with a weight of 70 kg, a total blood volume of 500 mL, an HCT of 0.4, a inlet flow rate (Qin) of 60 ml/min, and initial inlet blood versus AC ratio of 12, and an AC infusion rate of 1.0 mL/min/LTBV where LTBV is patient liters of total blood volume. The inlet flow rate is a combination of blood and AC. In at least one example embodiment, the AC ratio determines how fast the AC pump runs.

The algorithm is thus configured to determine when an inlet blood versus AC ratio should be increased to minimize an amount of AC introduced during an apheresis procedure. This algorithm ensure that a sufficient amount of AC is included throughout the disposables of the fluid components 300 to prevent coagulation. This may also lead to a sufficient citrate molarity being present within the apheresis machine to prevent coagulation of a patient's blood while also ensuring that side effects of citrate toxicity are minimized within a patient's body. In at least one example embodiment, an operator may verify the citrate molarity present within the apheresis machine during an apheresis procedure. In at least one example embodiment, the citrate molarity may be verified by visual inspection of the disposables or by alarms of the apheresis machine 100 indicating clotting within the disposables.

The systems and methods described herein provide improved apheresis procedures by improving safety via flow models that incorporate secondary devices and improving patient comfort and safety by maintaining desirable levels of citrate molarity during apheresis procedures. The improved flow models provide a more accurate flow model to be generated for a particular apheresis machine and/or procedure to ensure that an accurate volume and fluid flow is being determined during an apheresis procedure when secondary equipment is being used. The improved infusion rate for AC increases patient safety to reduce citrate toxicity while maintaining a necessary anticoagulation solution to complete an apheresis procedure. Thus, these systems and methods may provide improved apheresis procedures for medical practitioners, patients, and donors.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for determining a flow model for fluid within an apheresis machine, the method comprising:
determining a starting flow model for an apheresis procedure;
determining details of a secondary device, the secondary device configured to connect to the apheresis machine for the apheresis procedure; and
generating an updated flow model based on the starting flow model and the details of the secondary device.

2. The method of claim 1, wherein the secondary device is an external plasma treatment device.

3. The method of claim 1, wherein determining the starting flow model includes:
determining a fluid volume through each fluid line of a plurality of fluid lines of the apheresis machine;
determining a fluid volume in each fluid chamber of a plurality of fluid chambers of the apheresis machine; and
determining a plurality of fluids utilized during the apheresis procedure.

4. The method of claim 1, wherein determining details of the secondary device includes:
receiving a volume of the secondary device; and
determining an unknown fluid component within the secondary device when the secondary device is connected to the apheresis machine.

5. A method of performing an apheresis procedure using a flow model, the method comprising:
connecting a patient to an apheresis machine for the apheresis procedure, the apheresis machine including a secondary device;
generating a flow model for use during the apheresis procedure, the flow model incorporating the apheresis machine and the secondary device; and
initiating the apheresis procedure.

6. The method of claim 5, wherein the secondary device is a secondary plasma device.

7. The method of claim 5, wherein generating the flow model includes:
determining a starting flow model for the apheresis procedure;
determining details of the secondary device; and
generating an updated flow model based on the starting flow model and the details of the secondary device.

8. The method of claim 7, wherein determining the starting flow model includes:
determining a fluid volume through each fluid line of a plurality of fluid lines of the apheresis machine;
determining a fluid volume in each fluid chamber of a plurality of fluid chambers of the apheresis machine; and
determining a plurality of fluids utilized during the apheresis procedure.

9. The method of claim 7, wherein determining details of the secondary device includes:
receiving a volume of the secondary device; and
determining an unknown fluid component within the secondary device when the secondary device is connected to the apheresis machine.

10. The method of claim 5, further comprising:
tracking an amount of heparin throughout the apheresis machine and the secondary device during the apheresis procedure.

11. The method of claim 5, further comprising:
outputting a status of the apheresis procedure during operation of the apheresis machine.

12. A method for adjusting a ratio of inlet patient blood to anticoagulant (AC) during an apheresis procedure, the method comprising:
determining a citrate molarity for a first interval of a plurality of intervals;
during a current interval after the first interval, determining a citrate molarity for a previous interval;
determining if the citrate molarity for the previous interval is greater than a constant multiplied by the citrate molarity of the first interval; and
increasing the ratio of inlet patient blood to AC in response to the citrate molarity for the previous interval being greater than the constant multiplied by the citrate molarity of the first interval.

13. The method of claim 12, further comprising
maintaining the ratio of inlet patient blood to AC in response to the citrate molarity for the previous interval being less than or equal to the constant multiplied by the citrate molarity of the first interval.

14. The method of claim 12, wherein the constant is determined based on a time between intervals of the plurality of intervals.

15. The method of claim 12, wherein the citrate molarity for the previous interval is determined by dividing a sum of a number of moles of citrate in the inlet patient blood and a number of moles of citrate of the AC by a volume of an apheresis system divided by a change in time from a previous interval.

16. The method of claim 15, wherein the number of moles of citrate in the inlet patient blood is determined by multiplying a change in volume of the inlet patient blood from a previous interval by a plasma fraction and by a citrate molarity of the patient.

17. The method of claim 15, wherein the number of moles of citrate of the AC is determined by multiplying a change in volume of the AC from a previous interval by a citrate molarity of the AC.

18. The method of claim 15, wherein the apheresis system includes fluid components of an apheresis machine and blood components of the patient.

19. The method of claim 12, wherein the AC is ACD-A that includes citrate ions with a presumed half-life of 80 minutes.

20. The method of claim 12, wherein the ratio of inlet patient blood to AC is set by an operator prior to initiating the apheresis procedure.
